(19) United States
(12) Patent Application Publication
(10) Pub. No.: US 2002/0164301 A1
(43) Pub. Date: Nov. 7, 2002

(54) Methods for inhibiting atherosclerotic plaque formation

(76) Inventors: Jacques Van Snick, Brussels (BE); Johan Kuiper, Gouda (NL)

Correspondence Address: FULBRIGHT & JAWORSKI L.L.P., Mary Anne Schofield, Market Square, 801 Pennsylvania Avenue, N.W., Washington, DC, 20004-2615, US

(21) Appl. No.: 10/123,189

(22) Filed: Apr. 17, 2002

Related U.S. Application Data
(60) Provisional application No. 60/283,934, filed on Apr. 17, 2001.
(60) Provisional application No. 60/284,232, filed on Apr. 18, 2001.

(51) Int. Cl. A61K 38/20

(52) U.S. Cl. 424/85.2

(57) ABSTRACT

This invention relates to methods for inhibiting the initiation or progression of a pathologic disorder associated with atherosclerotic plaque formation comprising administering to a subject an amount of IL-9 sufficient to inhibit plaque formation and/or plaque progression and/or to promote plaque regression. The methods of this invention also relate to inhibiting the proliferation of smooth muscle cells in one or more arteries and to inhibiting the deposition and accumulation of fat and proteins in one or more arteries.

### RELATED APPLICATIONS

This application claims the benefit of U.S. Provisional Application No. 60/283,934 filed Apr. 17, 2002 and U.S. Provisional Application No. 60/284,232 filed Apr. 18, 2002 both incorporated herein by reference.

### FIELD OF THE INVENTION

This invention relates to methods for inhibiting the initiation or progression of a pathological condition associated with atherosclerotic plaque (plaques) formation. The invention also relates to methods for promoting the regression of plaques associated with atherosclerosis. The methods further relate to inhibiting the proliferation of smooth muscle cells in one or more arteries, and inhibiting the formation and expansion of fat and protein deposits within one or more arteries.

The method comprises administering an amount of IL-9 to a subject in need thereof wherein the amount of IL-9 is sufficient to prevent or inhibit the initiation of atherosclerotic plaques, inhibit the progression of plaques, and/or to promote the regression of plaques. In one embodiment the IL-9 is administered in an amount sufficient to inhibit the proliferation of smooth muscle cells in one or more arteries and/or to inhibit the formation and expansion of fat and protein deposits within one or more arteries. The methods of this invention also relate to administering IL-9 in an amount sufficient to inhibit the infiltration of monocytes, to inhibit activation of macrophages and to inhibit activation of macrophage derived foam cells within the atherosclerotic plaque.

### BACKGROUND OF THE INVENTION

Atherosclerosis is a general term for the thickening and hardening of arteries. Arteries comprise three main layers. The outside layer (the external elastic lamina or the adventitia) supports the artery and is composed predominantly of loose connective tissue. The middle layer (between the lamina elastica interna and externa), comprises predominantly smooth muscle (in mice this layer is very thin: 1-2 cells). The muscle cells provide for contraction and relaxation of the artery which controls the rate of blood flow. The inner layer of the artery is itself composed of three layers: an elastic layer (the internal elastic lamina), a basement layer (the intima) and an innermost layer (the endothelium). Atherosclerosis involves changes in the intima the inner layer of the artery.

Atherosclerosis is characterized by deposits of fatty substances, cholesterol, cellular waste products, calcium, proteins, deposits of extracellular matrix proteins, such as collagen, and other various specific proteins such as metallo proteases and the accumulation of intimal foam cells in medium and large sized arteries. Atherosclerosis appears to be a response to an initial injury to the inner lining of the artery and may be initiated by high serum cholesterol levels (Ross, R (1999) N. Engl. J Med. 340, 115-126). In response to high serum cholesterol levels in the blood, endothelial cells secrete factors which attract monocytes. Once the monocytes attach to the endothelium, they migrate through the endothelium and lodge just beneath the endothelial layer in the intima. After lodging in the artery, the monocytes mature to tissue macrophages and take up lipids and lipoproteins from the blood and become lipid filled foam cells. This process results in the formation of the initial atherosclerotic plaque The macrophage-derived foam cells release various mediators, e.g., cytokines and chemokines, free radicals, bioactive lipids, proteases, protease inhibitors and coagulation cascade components, which stimulate the migration and growth of smooth muscle cells. The smooth muscle cells may also take up lipids and transform into foam cells. During this process T lymphocytes infiltrate into the plaque and produce pro-inflammatory mediators thus contributing to the inflammatory process in the plaque. The initial lesion develops during the aforementioned processes through intermediate lesions to complex, advanced lesions (Ross, R (1999) N. Engl. J. Med. 340, 115-126) Lusis et al., “Atherosclerosis.” Nature 407, 233-241 (2000) Finally, damage to the endothelium, whether by the action of monocytes or other physical injury to the endothelium, attracts platelets. Often a blood clot forms and blocks the artery, stopping the flow of blood. Reducing the blood supply to the heart muscle may result in a heart attack. Reducing the blood supply to the brain may result in a stroke. Reducing the blood supply to a limb can result in gangrene.

Several reports suggest that atherosclerosis is a multifactorial disease with a large/major inflammatory component. (Ross, (1999) N. Engl. J. Med. 340, 115-126). Down regulation of the inflammatory component leads to a decreased level of atherosclerosis, e.g., adenoviral IL-10 gene therapy in low density lipoprotein (LDL) receptor knockout mice induces high levels of IL-10 and IL-10 significantly reduces the initiation of atherosclerosis (Terkeltaub, Artherioscler Thromb Vasc Biol 19:2823-2825 (1999); Pinderski et al, Arterioscler Thromb Vasc Biol 19:2847-2853 (1999); Mallat et al., Circ. Res, 85:1-8 (1999), and von der Thüsen, FASEB J., 15:2730-2732 (2000)).

Several models have been used to study atherosclerosis. Local lesion induction has been achieved by transluminal or extravascular arterial manipulation (Fishman et al., “Endothelial regeneration in the rat carotid artery and the significance of endothelial denudation in the pathogenesis of myointimal thickening”, Lab Invest., 32:339-351 (1975), and; Booth et al. “Rapid development of atherosclerotic lesions in the rabbit carotid artery induced by perivascular manipulation”, Atherosclerosis, 76:25 7-268 (1989)). Diet-induced hypercholesterolemina and genetically modified rabbits have also been used to study atherosclerosis (see, e.g., Finking and Hanke, “Nikolaj Nikolajewitsch Anitschkow (1885-1964) established the cholesterol-fed rabbit as a model for atherosclerosis research”, Atherosclerosis, 135:1-7 (1997); Fujiwara and Shiba, “Mechanisms of augmented vascular responses to histamine in atherosclerotic common carotid arteries”, Eur J Pharmacol., 258:195-201 (1994), and; Matthys et al., “Local application of LDL promotes intimal thickening in the collared carotid artery of the rabbit” Arterioscler Thromb Basc Biol., 17:2423-2429 (1997)). Mouse models for atherosclerosis include, e.g., LDL receptor knockout mice described by Ishibashi et al. infra, apolipoprotein E knockout mice (apoE−/−) described by Nakashima et al. infra and apolipoprotein E3-Leiden transgenic mice described by van den Maagdenberg infra ((Ishibashi et al., “Massive xanthomatosis and atherosclerosis in cholesterol-fed low density lipoprotein receptor-negative mice”, J Clin Invest. 93:1885-1893 (1994); Nakashima et al. “ApoE-deficient mice develop lesions of all phases of atherosclerosis throughout the arterial tree”, Arterioscler Thromb., 14:133-140 (1994), and; van den Maagdenberg et al., “Transgenic mice carrying the apolipoprotein E3-Leiden gene exhibit hyperlipoproteinemia”, J Biol Chem., 268:10540-10545 (1993)).

Using the LDL receptor deficient mice we established a rapid model for atherosclerosis, which was used in these studies (von der Thüsen, et al. Circulation, 103, 1164-1170 (2001)). These models have been useful in analyzing the role of diet, environmental factors, and genetics in the initiation and progression of atherosclerosis. Described herein are the effects of IL-9 on the initiation, progression and regression of plaques associated with atherosclerosis and the effect of IL-9 on the proliferation of smooth muscle cells and the formation and enlargement of fat and/or protein deposits in one or more arteries.

### BRIEF DESCRIPTION OF THE FIGURES

FIGS. 1A-D depict the effect of intraperitoneal administered IL-9 (lug/mouse/day) on collar-induced atherosclerosis in LDL receptor deficient male mice.

FIG. 2 depicts the effect of IL-9 on atherosclerosis (2A, plaque size μm²; 2B, median size, μm²) in LDL receptor deficient female mice treated for 4 weeks with daily injections of 1 μg IL-9. The extent of atherosclerosis was determined in the carotid artery after collar placement.

FIG. 3 depicts the effect of IL-9 on TNF-α production in whole blood of LDL receptor deficient mice treated for 5 days with 1 μg IL-9 per day. The TNF-α production ex vivo was determined in response to increasing amounts of lipopolysaccharide (LPS).

FIG. 4 depicts the extent of atherosclerosis (4A, plaque size, μm²; 4B, median size, μm²) in LDL receptor deficient mice immunized with IL-9 ovalbumin conjugates (IL-9-OVA). The extent of atherosclerosis was determined in the carotid artery after collar placement.

### SUMMARY OF THE INVENTION

This invention relates to methods for preventing or inhibiting the progression of a pathologic condition associated with atherosclerotic plaque formation. Pathologic conditions associated with atherosclerotic plaque formation include e.g., atherosclerosis, stroke, heart attacks, unstable angina and gangrene due to blocked blood vessels. The methods of this invention also relate to inhibiting the initiation of atherosclerotic plaques, inhibiting the progression of plaques, or promoting the regression of plaques associated with atherosclerosis in a subject in need thereof. The methods are useful for the treatment and prevention of vulnerable plaques, unstable plaques or rupture prone plaques (Stary, et al., Arterioscl. Thromb., 14, 840-856 (1994) and Stary, et al., Arteriscl. Thromb. Vasc. Biol., 20, 1177-1178 (2000)). This invention relates to methods useful for inhibiting the formation and enlargement of fat and protein deposits and to inhibiting the proliferation of smooth muscle cells in one or more arteries in an animal.

The methods comprise administering an amount of IL-9 to a subject in need thereof wherein the amount is sufficient to prevent the formation of an atherosclerotic plaque, to inhibit the progression of the plaque and eventually to promote the regression of an atherosclerotic plaque. The administration of IL-9 inhibits the initiation or progression of atherosclerotic plaque formation, which is manifested as a reduction in the average size of plaques as compared to a control that is not treated with IL-9. Also an embodiment of this invention is a method for promoting the regression of plaques by administering an amount of IL-9 to promote regression of plaques. This may be manifested in a reduction in the size or number of already existing plaques.

In one embodiment of the invention the amount of IL-9 administered to the subject is sufficient to inhibit or prevent the proliferation of foam cells and smooth muscle cells and monocytes or monocyte derived macrophages in arteries, and/or to inhibit the formation of fat deposits or protein deposits in one or more arteries. Preferably the amount of IL-9 is sufficient to inhibit the initiation or progression of plaques or to promote the regression of plaques, e.g., vulnerable plaques, unstable plaques and rupture prone plaques.

Preferably the IL-9 is an autologous IL-9, e.g., an IL-9 of the species of the subject to which it is administered, e.g., if the subject is a human the administered IL-9 is a human IL-9 or if the subject is a dog the administered IL-9 is a dog IL-9. The IL-9 may be isolated from a natural source, e.g., from serum or it may be produced recombinantly. Preferably the IL-9 is produced recombinantly. Those of skill in the art appreciate that there are a variety of commercially available sources for cytokines such as IL-9 and that there are a variety of methods available that are suitable for producing a recombinant IL-9 that is useful in the methods of this invention. See, e.g., Druez, et al., “Functional and biochemical characterization of mouse P40/IL-9 receptors” J. Immunol., 145:2494-2499(1990) for methods for producing a murine IL-9 in insect cells under the control of a baculovirus promoter. IL-9 produced in insect cells under the control of baculovirus promoters has a short half life, which may be the consequence of a high mannose content and lack of terminal sialic acid. IL-9 isolated from the serum of IL-9 transgenic mice, display a substantially stronger effect than the baculovirus produced IL-9, e.g., 50 ηg of IL-9 isolated from the serum of the transgenic mice display a stronger effect than 4 μg baculovirus produced IL-9.

Also useful in the methods of this invention is a conjugate of IL-9 and a conjugation partner e.g. polyethylene glycol. Preferably the conjugation partner does not promote an immune response to itself or to the IL-9 such that repeated treatments with IL-9 or the conjugated IL-9 are possible. Conjugates of IL-9 and polyethylene glycol have been shown to increase the activity of IL-9 in vitro. Methods for preparing conjugates of cytokines and polyethylene glycol are well known in the art. See, e.g., Cunningham-Rundles et al., “Long-term low-dose IL-2 enhances immune function in common variable immunodeficiency”, Clin. Immunol, 100(2):181-90 (August, 2001) and Meyers et al., “A phase I study including pharmacokinetics of polyethylene glycol conjugated interleukin-2”, Clin. Pharmacol. Ther., 49(3):307-13 (March, 1991).

Other forms of IL-9 are also useful in the methods of this invention, e.g., any fragment of IL-9 that binds to cellular IL-9 receptors and induces an IL-9 response by those cells would be suitable for use in the methods of this invention. The binding of an IL-9 to IL-9 receptor may be assayed by any method known in the art. The induction of a response by a suitable IL-9 fragment may be determined by a variety of assays, e.g., by assaying for proliferation of PHA plus IL-4 stimulated human lymphoblast lines (Yang et al., Blood, 74:1880-1884 (1989, incorporated herein by reference).

IL-9 may be administered to the subject with any pharmaceutically acceptable carrier and in any pharmaceutically acceptable manner. For example, IL-9 may be administered e.g., intramuscularly, intradermally, intra-arterially, subcutaneously, intraperitoneally, intravenously and intraventricularly. Preferably, IL-9 is administered subcutaneously.

Gene therapy methods for delivering IL-9 to a subject in need thereof to inhibit the initiation and progression of artherosclerotic plaques are also contemplated herein. A nucleic acid molecule encoding an IL-9 may be introduced into cells ex vivo, wherein harvested cells are transformed with the IL-9-encoding nucleic acid molecule and then the transformed cells reintroduced into a subject, or the polynucleotide may be introduced into cells in vivo via a vector. For example, an IL-9 encoding sequence can be incorporated into naked DNA vectors, e.g., plasmids, and introduced into cells by using e.g., cationic lipids or liposomes. Alternatively the nucleic acid molecule encoding IL-9 may be introduced into cells, in vivo and ex vivo via viral vectors, e.g., adenoviral vectors, adeno associated viral vectors, lentiviral vectors or retroviral vectors, and the vectors, and expressed at levels that are sufficient to inhibit the initiation or progression of atherosclerotic plaque formation. The vectors may be introduced into a subject directly, e.g., by injection of the vectors either locally or systemically and the vectors may be designed for constitutive or inducible IL-9 expression and the vectors may be designed for their transient presence, e.g., not incorporated within the genome of a cell, or a their permanent presence, e.g., integrated into a cell genome. Gene therapy has been used to introduce a variety of therapeutic genes into subjects in need thereof, see for example, Tolstoshev, Ann. Rev. Pharm. Toxicol., 32:573-596 (1993); Morgan et al. Ann Rev. Biochem 62:191-217 (1993) for a review and also U.S. Pat. Nos. 6,538,915 issued Mar. 19, 2002, 5,981,501 issued Nov. 9, 1999 and 5,656,465 issued Aug. 12, 1997 all incorporated herein by reference. Gene therapy vectors are also commercially available from different laboratories, e.g., Chiron, Inc., Emeryville, Calif.; Genetic Therapy, Inc., Gaithersburg, Md.; Genzyme, Cambridge, Mass.; Targeted Genetics, Seattle, Wash., and; Viagene, San Diego Calif.

IL-9 may be administered monthly, weekly or daily for a predetermined period of time.

Suitable carriers include but are not limited to pharmaceutically acceptable diluents of various buffer content (e.g., Tris-HCl, acetate, phosphate), pH and ionic strength; and may include additives such as detergents and solubilizing agents (e.g., Tween 80, Polysorbate 80), antioxidants (e.g., ascorbic acid, sodium metabisulfite), and preservatives (e.g., Thimersol, benzyl alcohol) and bulking substances (e.g., lactose, mannitol). IL-9 may be incorporated into particulate preparations of polymeric compounds such as polylactic acid, polyglycolic acid, etc. or into liposomes. Hylauronic acid may also be used. Such compositions may influence the physical state, stability, rate of in vivo release, and rate of in vivo clearance. See, e.g., Remington's Pharmaceutical Sciences, 18th Ed. (1990, Mack Publishing Co., Easton, Pa. 18042) pages 1435-1712 which are herein incorporated by reference.

Those of skill in the art appreciate that the amount of IL-9 sufficient to prevent, inhibit or promote regression of plaques associated with atherosclerosis, or sufficient to inhibit smooth muscle cell proliferation, or inhibit the deposition and accumulation of fats and proteins in one or more arteries can readily be determined using routine methods available in the art. Preferably the effective amount is about 40 ug/kg body weight, equivalent to about 2.1-3.2 mg per patient. Those of skill in the art are well aware of methods useful for detecting arterial plaques and assaying their size and progression or regression (von der Thüsen et al., “Induction of Rapid Atherogenesis by Perivascular Carotid Collar Placement in Apolipoprotein E-Deficient and Low-density Lipoprotein Receptor-Deficient Mice”, Circulation 103:1164-1170 (2001) incorporated herein by reference). Thus one of skill in the art could assay the size of arterial plaques before and after treatment with IL-9 to determine the dose of IL-9 needed to be increased or decreased. A decrease in the size or number of arterial plaques would indicate that a suitable dose of IL-9 is being administered.

The methods of this invention are applicable to any subject in need thereof. The subject in need thereof may be any mammal which has a predilection for developing atherosclerosis, for example a subject who has a family history of developing atherosclerotic plaques, a subject having Familial Hypercholesteremia, which is an inherited disorder that leads to high cholesterol levels, or a subject having high plasma cholesterol levels without a family history of high cholesterol, or any mammal already having atherosclerotic plaques in one or more arteries. By inhibiting the initiation and progression of plaque formation, the initiation and progression of pathologic conditions associated with plaque formation, e.g., atherosclerosis, stroke, heart attacks, unstable angina and gangrene associated with a blocked blood vessel, are also inhibited. Those of skill in the art are well aware of methods used to determine if a subject harbors atherosclerotic plaques or has an increased chance of developing atherosclerotic plaques (see, e.g., Williams Hematology, 2d edition, Beutler et al. eds., (2001), chapter 30;Ross, N. Engl. J Med. 340, 115-126 (1999), Lusis, “Atherosclerosis.” Nature 407, 233-241 (2000) (all incorporated herein by reference). The atherosclerotic plaques may be end stage plaques, e.g., vulnerable plaques, unstable plaques or rupture prone plaques or any combination thereof. Preferably the mammal is a human, a mouse, a guinea pig, a cat, a dog, a horse, a cow or a pig. More preferably the subject is a human.

Also an aspect of the invention is a method for inducing the production of IL-9 in a subject in need thereof, wherein IL-9 production or activity is induced to a level that is sufficient to prevent the formation of atherosclerotic plaques, to inhibit the progression of plaques, and/or to promote the regression of plaques associated with atherosclerosis. In another embodiment of the invention IL-9 production or activity is induced to sufficient levels to prevent the proliferation of smooth muscle cells in arteries and to prevent the deposition of fat and proteins in arteries. Such methods comprise, e.g., administering an agent that promotes the synthesis of IL-9, or enhances the activity of IL-9, to the subject. Also envisioned is the production of IL-9 from a gene introduced into a subject via gene therapy using either viral vectors, e.g., adenoviral vectors, lentiviral vectors or retroviral vectors or naked DNA vectors, e.g., plasmids.

Because administration of IL-9 reduces plaque formation in the mouse model, a low level of IL-9 as compared to a predetermined control level may be indicative of a subject's predilection for the development of atherosclerotic plaques and could be used to suggest measures that would decrease the risk of developing plaques, e.g., a change in diet to one that is low in cholesterol or increasing the subjects level of exercise. Thus, a further aspect of this invention are methods for assessing the predilection of a subject for the development of atherosclerotic plaques by assaying the subject for a reduced level of IL-9 wherein a reduced level of IL-9, as compared to a predetermined control level, is indicative of a predilection of said subject for the development of atherosclerotic plaques. Levels of IL-9 may be determined in a variety of assays. For example, one could measure IL-9 production by assaying peripheral blood lymphocyte in vitro response to polyclonal stimulation with anti-CD3, or PHA or with LDL or a modified LDL.

Also an aspect of this invention is the use of an IL-9 in the manufacture of a medicament for treating a pathologic disorder associated with arterial plaque formation in a subject in need thereof. Such pathological disorders include, e.g., atherosclerosis, heart attack, unstable angina, stroke or gangrene due to blocked blood vessel. Another aspect of this invention is the use of a vector comprising a sequence encoding IL-9 in the manufacture of a medicament for use in gene therapy of a pathologic disorder associated with arterial plaque formation. The vectors may be a viral vector e.g., a retroviral vector, an adenoviral vector, an adeno associated viral vector or a lentiviral vector or a nucleic acid vector e.g., a plasmid. The vectors may be designed such that they are for temporary expression of IL-9, constitutive expression of IL-9 or permanent expression of IL-9. The IL-9 may be a naturally occurring IL-9, an autologous IL-9, a recombinant IL-9, or an IL-9 conjugate, e.g., pegylated IL-9, wherein the conjugation partner does not promote antibody production to itself or to the IL-9. The IL-9 may also be a fragment of IL-9 that binds to cellular IL-9 receptors and induces an IL-9 response by those cells would be suitable for use in the methods of this invention. The IL-9 may be produced in culture, for example in mammalian cell culture or in insect cell culture. The subject in need thereof may be a mammal, e.g., a mouse, a rat, a guinea pig, a cat, a dog, a pig, a cow, a horse or a human. A subject in need thereof may be one who displays a predilection for developing arterial plaques, has a family history of developing atherosclerotic plaques, a subject having Familial Hypercholesteremia, which is an inherited disorder that leads to high cholesterol levels, or a subject having high plasma cholesterol levels without a family history of high cholesterol, or one who already has a plaque, e.g., a vulnerable plaque, an unstable plaque or a rupture prone plaque, in one or more arteries. A subject in need thereof may have reduced levels of LDL receptors or apolipoprotein E.

### DETAILED DESCRIPTION OF THE INVENTION

### EXAMPLE 1

LDL receptor deficient mice, transgenic mice developed essentially as described in Ishibashi et al., “Massive Xanthomatosis And Atherosclerosis In Cholesterol Fed Low Density Lipoprotein Receptor-Negative Mice”, J. Clin. Invest., 93:1885-1893 (1994), incorporated herein by reference, were used in these examples. The LDL deficient mice are currently used as a model for the development of atherosclerosis (see von der Thüsen et al., Circulation (2001) supra).

Male LDL receptor deficient mice were put on a cholesterol rich diet (type W diet containing 0.25% cholesterol, 15% cocoa butter). After 14 days, collars were placed around the left and the right carotid artery (as described in von der Thüsen Circulation 2001, supra). The mice were then treated with IL-9 with daily intraperitoneal injection with 1 μg baculovirus recombinant IL-9 (Druez, et al., J. Immunol., 145:2494-2499(1990) incorporated herein by reference) per mouse per day from day 21 to day 56. Control animals received daily injections with vehicle alone (PBS containing 1% autologous mouse serum).

Body weight, cholesterol levels and lipoprotein profile were monitored throughout the experiment. At the end of the experiment (day 56 after the last dose of IL-9), animals were anesthetized and exsanguinated by femoral artery transection. In situ perfusion fixation through the left cardiac ventricle was performed by PBS instillation for 15 minutes, followed by constant-pressure infusion (at 80 mm Hg) of 10% neutral buffered formalin for 30 minutes. Subsequently, both carotid bifurcations and common carotid arteries were removed. No differences were observed between the body weight of IL-9-treated and vehicle-treated mice. In addition, IL-9 treatment did not affect the cholesterol levels as compared to the control mice. Throughout the experiments the mice, regardless of treatment, maintained a level of approximately 3000 mg cholesterol/dl. IL-9 treatment did not alter the lipoprotein profile of the treated mice as compared to the control mice (80% of the total cholesterol is recovered in both groups in the VLDL fraction).

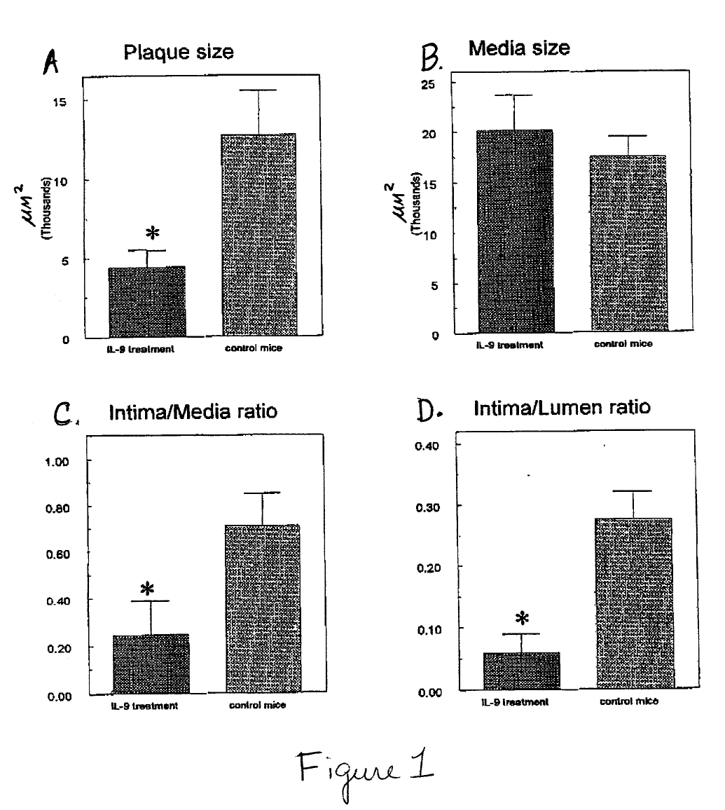

The collar-induced atherosclerosis in treated and untreated mice was assayed by determining plaque size (surface area at the point where the size/area of the plaque is maximal) media size (between the intima (plaque) and the smooth muscle layer), intima/media ratio and intima/lumen ratio (FIG. 1 A-D) essentially as described in von der Thüsen (Circulation 2001 supra). Briefly, hematoxylin and eosin-stained sections were assessed in cross-section at 3 levels: 0.5 mm proximal, in the mid-section and 0.5 mm distal to the collar. The intimal surface area was calculated by subtracting the patent lumen area from the area circumscribed by the internal elastic lamina. The medial surface area was defined as the area between the internal elastic lamina and the external elastic lamina. The intima/media ratio and the intima/lumen ratio were determined by dividing the intimal area by the medial area and the total area confined by the internal elastic lamina, respectively.

The results are set forth in FIGS. 1A through 1D and indicate that IL-9 significantly reduced plaque size without effect on the size of the media. These results clearly demonstrate that daily treatment of mice with IL-9 significantly reduces the initiation of atherosclerosis.

### EXAMPLE 2

Example 1 was repeated in female LDL receptor deficient mice and the effects of IL-9 on atherosclerotic plaque formation was evaluated. On Day 1, two groups of mice (Group A (IL-9 treated, n=9) and Group B (control, n=8)) were put on a western type diet containing 0.25% cholesterol and 15% cocoa butter. At Day 15 collars were placed around the left and right carotid artery (as described by von der Thüsen et al., Circulation (2001) supra). From Day 16 through Day 42 the Group A mice were injected daily (intra-peritoneal) with 1 μg baculovirus produced IL-9 dissolved in 100 μl of PBS (containing 1% normal autologous mouse serum). The Group B control mice received a daily intra-peritoneal injection of 100 μl of PBS (containing 1% normal autologous mouse serum).

At Day 42, both groups of mice were anaesthetized and exsanguinated by femoral artery transection, and in situ perfusion fixation through the left cardiac ventricle was performed by PBS instillation for 15 minutes, followed by constant-pressure infusion (at 80 mm Hg) of 10% neutral buffered formalin for 30 minutes. Subsequently, both carotid bifurcations and common carotid arteries were removed. Formalin fixation was omitted for arteries that were to be stained for von Willebrand Factor “vWF”; these were immediately snap-frozen in liquid nitrogen after having been embedded in OCT compound (Tissue-Tek; Sakura Finetek), whereas the remaining arteries were left in 10% formalin overnight before freezing. The specimens were stored at −20° C. until further use. Transverse 5-mm cryosections were prepared in a proximal direction from the carotid bifurcation and mounted in order on a parallel series of slides.

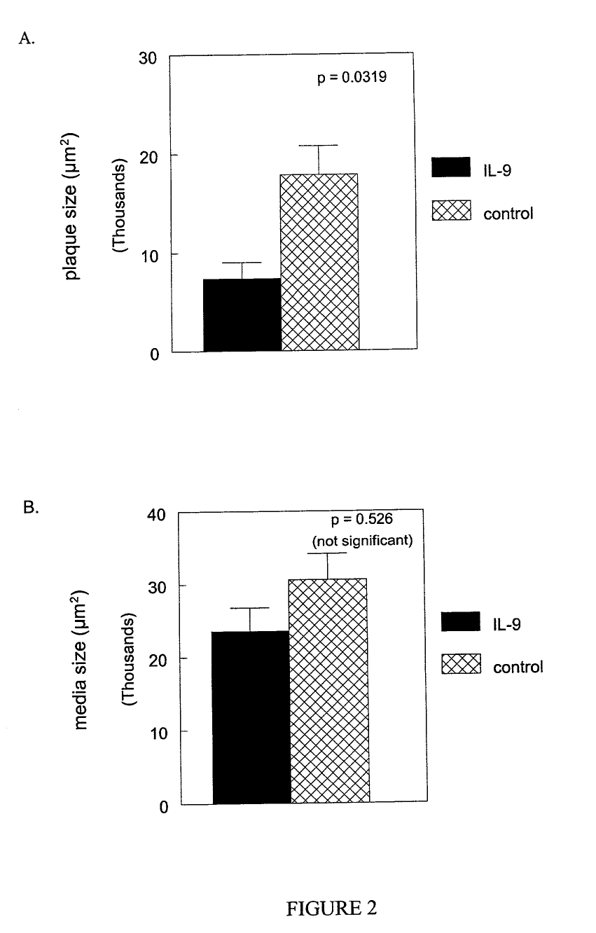

FIG. 2 depicts the effects of baculovirus-produced IL-9 on the development of atherosclerotic plaques. The mice of Group A, which were treated with IL-9, showed a clear diminishment in the extent of atherosclerosis. The significant reduction in the extent of atherosclerosis was 58.6% in comparison to the control group (p<0.05).

### EXAMPLE 3

The effect of IL-9 on TNF-A production by blood monocytes in response to LPS was determined in a whole blood assay.

Mice (Group A: IL-9 treated, n=9)) received a daily intra-peritoneal injection of recombinant IL-9 dissolved in 100 μl of PBS (containing 1% normal autologous mouse serum) for five days. Control mice (Group B, n=8) received a daily i.p. injection of 100 μl of PBS (containing 1% normal autologous mouse serum) for five days. At day 5 blood was collected from the tail vein of all mice. Whole blood was obtained by tail vein transection and diluted 25 fold in Dulbecco's modified Eagle's medium supplemented with L-glutamine, penicillin and streptomycin, which contained varying concentration so lipopolysaccharide (Re 595, List Biological Laboratories, Campbell, Calif.). Following incubation overnight at 37° C., 50 μl of the supernatent was analyzed for TNF-α content by ELISA.

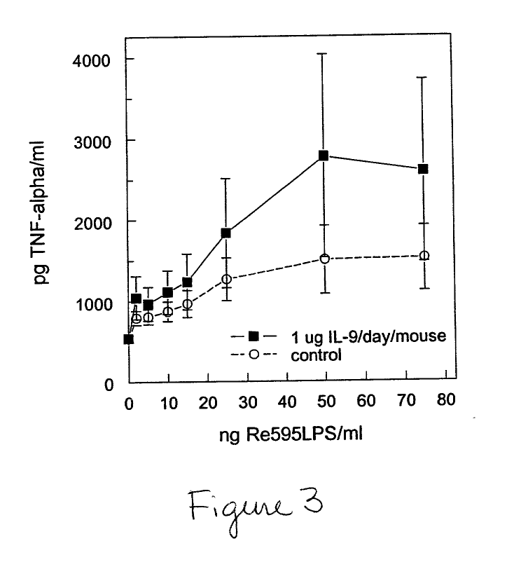

The results are depicted in FIG. 3. The TNF-α production in the whole blood assay after LPS stimulation was not significantly different in the IL-9 treated animals as compared to the control treated animals.

### EXAMPLE 4

The effect of endogenous interleukin 9 on atherosclerosis was also assayed by vaccinating mice with IL-9 ovalbumin conjugates (IL-9-OVA) prior to placing the mice on a diet containing 0.25% cholesterol and 15% cocoa butter.

On Day 1, 10 female LDL receptor mice (Group A) were vaccinated in both footpads using 1 μg of IL-9-ovalbumin conjugate in the presence of complete Freund's adjuvant as described by Richard et al., (“Anti-IL-9 vaccination prevents worm expulsion and blood eosinophilia in Trichuris muris-infected mice”, PNAS 97 767-772 (2000) incorporated herein by reference). Control mice were 10 female LDL receptor mice vaccinated with ovalbumin in the presence of complete Freund's adjuvant (Group B).

On Days 15, 29 and 43, the Group A mice were vaccinated with 1 μg of IL-9-ovalbumin conjugate in the presence of incomplete Freund's adjuvant. On Days 15, 29 and 43, the control Group B mice were vaccinated with ovalbumin in the presence of incomplete Freund's adjuvant.

On Day 57 the two groups of mice were put on a western type diet (0.25% cholesterol, 15% cocoa butter) and assayed for the production of IL-9 specific antibodies. Anti-IL-9 titers of the vaccinated mice were tested in a TS1 assay. The titers are the reciprocal dilutions of the sera that produce 50 % inhibition of IL-9 (50 pg/ml). The only Group A mice that were included in the experiment were those that had a significant level of anti-IL-9 antibodies (6/10 mice). The control mice vaccinated with OVA did not produce IL-9 antibodies.

Two weeks later (Day 71) collars were placed around the left and right carotid artery (as described by von der Thüsen et al. 2001 supra) of the control mice and the mice with the significant levels of anti-IL-9 antibody.

On Day 113, both groups of mice were anaesthetized, and in situ perfusion fixation through the left cardiac ventricle was performed by PBS instillation for 15 minutes, followed by constant-pressure infusion (at 80 mm Hg) of 10% neutral buffered formalin for 30 minutes. Subsequently, both carotid bifurcations and common carotid arteries were removed. Formalin fixation was omitted for arteries that were to be stained for vWF; these were immediately snap-frozen in liquid nitrogen after having been embedded in OCT compound (Tissue-Tek; Sakura Finetek), whereas the remaining arteries were left in 10% formalin overnight before freezing. The specimens were stored at −20° C. until further use. Transverse 5-mm cryosections were prepared in a proximal direction from the carotid bifurcation and mounted in order on a parallel series of slides.

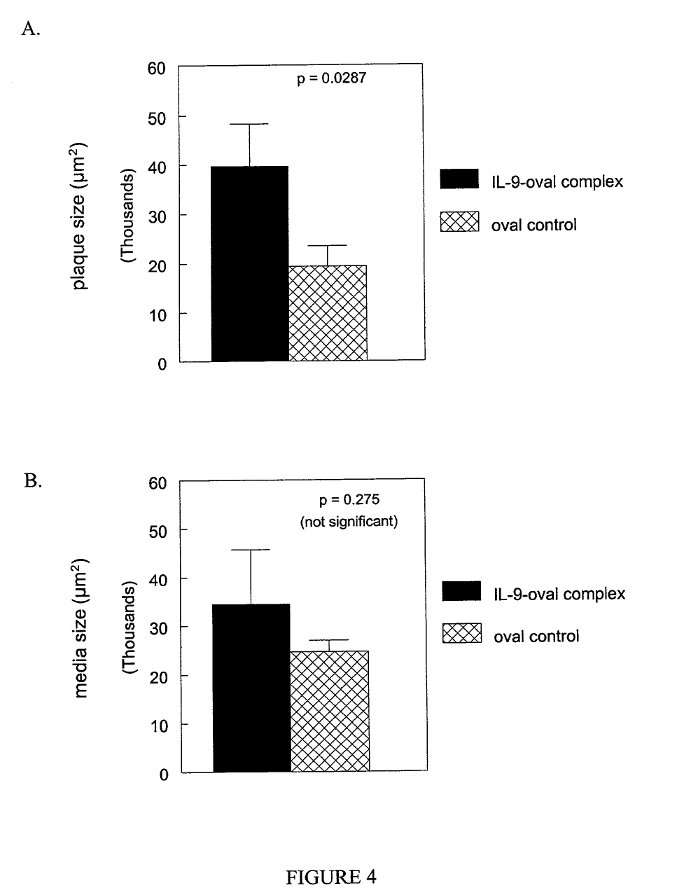

FIG. 4 demonstrates that the Group A mice, which were vaccinated with IL-9-OVA conjugates and had significant levels of IL-9 specific antibodies, had a clear increase in the extent of atherosclerosis. The level of atherosclerosis was more than double (2.05 fold) the level in control mice which were vaccinated ovalbumin (p<0.05).

The results set forth herein demonstrate that administration of IL-9 to a subject inhibits formation and progression of atherosclerotic plaques. The increase in atherosclerosis as a result of IL-9-OVA immunization demonstrates that endogenous IL-9 plays a role in inhibiting atherosclerosis and that IL-9 does not prevent the subsequent production of TNF by blood monocytes in response to LPS in vitro.

The terms and expressions which have been employed are used as terms of description and not of limitation, and there is no intention in the use of such terms and expressions of excluding any equivalents of the features shown and described or any portions thereof, it being recognized that various modifications are possible within the scope of the invention.

We claim:

1. A method for inhibiting initiation or progression of a pathological disorder associated with atherosclerotic plaque (plaque) formation in a subject in need thereof comprising administering to a subject in need thereof an amount of an IL-9 sufficient to inhibit initiation or progression of an atherosclerotic plaque thereby inhibiting the initiation or progression of the pathological disorder.

2. The method of claim 1, wherein said subject is a mammal.

3. The method of claim 2, wherein said mammal is a human.

4. The method of claim 1, wherein said IL-9 is a recombinant IL-9.

5. The method of claim 1, wherein said IL-9 is a pegylated IL-9.

6. The method of claim 1, wherein said IL-9 is a fragment of IL-9 sufficient to bind to IL-9 receptor.

7. The method of claim 1, wherein the IL-9 is administered to the subject in need thereof prior to plaque formation.

8. The method of claim 1, wherein the IL-9 is administered to the subject in need thereof after a plaque has formed in one or more arteries.

9. The method of claim 8, wherein the plaque is a vulnerable plaque, an unstable plaque or a rupture prone plaque.

10. The method of claim 1, wherein said subject in need thereof has reduced levels of LDL receptors or apolipoprotein E.

11. The method of claim 1, wherein the IL-9 is administered in an amount sufficient to inhibit the proliferation of smooth muscle cells in one or more arteries.

12. The method of claim 1, wherein the IL-9 is administered in an amount sufficient to inhibit the deposition of fat or proteins, or both, in one or more arteries.
